(19) United States
(12) Patent Application Publication
(10) Pub. No.: US 2021/0059970 A1
(43) Pub. Date: Mar. 4, 2021

(54) NUTRITIONAL SUPPLEMENTS AND METHODS OF NUTRITIONAL SUPPLEMENTATION AFFECTING MENTAL WELLNESS AND/OR MOOD

(71) Applicant: Amare Global, Irvine, CA (US)

(72) Inventor: Shawn M. TALBOTT, Draper, UT (US)

(73) Assignee: Amare Global, Irvine, CA (US)

(21) Appl. No.: 17/012,704

(22) Filed: Sep. 4, 2020

Related U.S. Application Data
(60) Provisional application No. 62/895,906, filed on Sep. 4, 2019.

(51) Int. Cl. A61K 31/202; A61K 36/53

(52) U.S. Cl. CPC A61K 31/202; A61K 36/53

(57) ABSTRACT

Nutritional supplements and methods of nutritional supplementation that affect mental wellness and/or mood are provided. The supplements and methods of supplementation improve mental wellness and/or mood by supporting both brain and gut health and relaxation by the oral co-administration of eicosapentaenoic acid, docosahexaenoic acid, and lavender oil.

### BACKGROUND

The following relates generally to nutritional supplements and to methods of nutritional supplementation of subjects. More specifically, the following relates to nutritional supplements affecting mental wellness and/or mood in subjects, and to methods of supplementation of such subjects to improve mental wellness and mood.

Mood disorders are a daily struggle for many adults and children, who face each day with a lowered emotional state for weeks at a time. These disorders often affect behavior and may reduce a sufferer's ability to face daily activities regarded by others as routine, such as work, school, or normal interactions and pressures at home.

Common mood disorders include depression and bipolar disorder. These illnesses have a variety of causes ranging from environmental factors to biological triggers and may be familial as well. Symptoms include sadness, diminished energy or vigor, reduced self-esteem, diminished interest in previously-cherished activities, changes in appetite and/or weight, and disruption of sleep. Thought patterns may become disordered or suicidal, and behaviors often change significantly as well.

Although a variety of pharmaceutical and talk therapy options exist to address these disorders, some individuals experiencing mood disorders struggle to find adequate reduction of symptoms, and/or suffer from the often significant side-effects of the medications.

Many individuals would benefit from nutritional supplements and methods of nutritional supplementation which improve mental wellness, and support mood. In some cases, it would be beneficial to provide nutritional supplements and methods of nutritional supplementation which are beneficial to mental wellness by nourishing brain and gut health.

### SUMMARY

This application discloses improved supplements and methods of nutritional supplementation that affect mental wellness and/or mood. Generally, the described techniques provide for unique nutritional supplements and methods for their use to improve mental wellness and/or mood by supporting both brain and gut health.

A method for improving mental wellness and/or mood in a subject is described. The method may include administering to the subject an effective amount of a composition including EPA, DHA and lavender oil. In some instances, the methods include administering to the subject a composition in which the EPA and DHA are present in a 5:1 ratio.

In some examples of the present disclosure the compositions may include approximately 1500 mg of fish oil per serving, with 975 mg of total omega-3 fatty acids. This may provide approximately 750 mg of EPA and approximately 150 mg of DHA with 75 mg of other omega-3s.

In some examples of the present disclosure, the methods of supplementation described herein further include the step of identifying a subject in need of improvement of mental wellness and/or mood. In some of these methods, identifying a subject in need of improvement of focus and/or mental performance may include identifying a subject in need of at least one of improvement of mood, reduction of depression, increased energy or vigor, and/or improved self-esteem.

The present disclosure further provides nutritional supplements for improving mental wellness and/or mood in a subject. Such nutritional supplements may include an effective amount of a composition including EPA, DHA and lavender oil. In some instances, the supplements include a composition in which the EPA and DHA are present in a 5:1 ratio.

### DETAILED DESCRIPTION

As used herein, treatment means any manner in which the symptoms or pathology of a condition, disorder or disease are ameliorated or otherwise beneficially altered. Treatment also encompasses any pharmaceutical use of the compositions herein.

The supplement compositions of the present disclosure may be administered in a variety of suitable dosage forms, including, without limitation, tablets, capsules, granules, powders, liquids, liposome inclusions, ointments, gels, external powders, sprays, inhalable powders, injectable preparations (solutions, suspensions, emulsions, solids to be dissolved when used, etc.), eye drops, eye ointments, suppositories, and the like can be selected appropriately depending on the administration method, and the compositions of the present disclosure can be accordingly formulated. Formulation in general is described in references including Comprehensive Medicinal Chemistry, Volume 5, Editor Hansch et al, Pergamon Press 1990.

As used herein, any range set forth is inclusive of the end points of the range unless otherwise stated.

As used herein, “effective amount” refers to an amount of a substance which is sufficient to achieve its intended purpose or effect. Various biological factors may affect the ability of a delivered substance to perform its intended task. Therefore, an “effective amount” may be dependent on such biological factors. An effective amount of a compound for treating a disorder is an amount that is sufficient to ameliorate, or in some manner reduce a symptom or stop or reverse progression of a condition. Such amount may be administered as a single dosage or may be administered according to a regimen whereby it is effective. The achievement of therapeutic effects may be measured by a physician or other qualified medical personnel using evaluations known in the art, (for example with testosterone supplementation therapy, physical examination, blood and saliva tests may be used), it is recognized that individual variation and response to treatments may make the achievement of therapeutic effects a subjective decision.

Further, determination of the effectiveness of the amount is well within the knowledge and ability of one of ordinary skill in the art.

As used herein, “administration,” and “administering” may be used interchangeably, and refer to the act of presenting, applying, or introducing a drug to a subject in order to achieve a desired physiological or psychological response.

As used herein, amelioration of the symptoms of a particular disorder by administration of a particular pharmaceutical composition refers to any lessening, whether permanent or temporary, lasting or transient that can be attributed to or associated with administration of the composition.

It is well understood that mood disorders are common and debilitating struggle for many who experience a diminished or depressed emotional state, in some cases for weeks at a time. These disorders affect behavior, and often reduce the ability of one suffering from them to successfully complete routine tasks and activities.

Depression and bipolar disorder are thought to be triggered by any of a variety of environmental, biological, and/or genetic factors. The sadness, diminished energy, reduced self-esteem, diminished interest, changes in appetite and/or weight, and disruption of sleep experienced by those suffering from mood disorders can be profoundly disruptive and difficult to successfully treat or prevent, leaving a need for alternative therapeutic options.

Many individuals would benefit from nutritional supplements and methods of nutritional supplementation which improve mental wellness, and support mood. In some cases, it would be beneficial to provide nutritional supplements and methods of nutritional supplementation which are beneficial to mental wellness by nourishing brain and gut health.

It is thought that eicosapentaenoic acid (“EPA”) may be used to support general brain health by managing inflammation. It is further thought that docosahexaenoic acid (“DHA”) may be used to support brain health by enhancing cognition and neuronal growth and/or development. Each of these compounds addresses brain health solely through affecting brain tissue, however. Inhaled lavender is often used as an aromatherapeutic substance to reduce stress and calm a subject.

It has been determined that combining the brain supportive actions of EPA and DHA in a specific 5:1 ratio with orally-ingested lavender acts in a unique and unexpected way to relax the smooth muscle of the gastrointestinal tract. This smooth muscle relaxation combines with the effects of the EPA and DHA to exert an overall calming and mood-elevating effect on a subject. This effect is possible due to the gut-brain-axis found in humans.

As a result, methods for improving mental wellness and/or mood in a subject are described herein. These methods may include orally administering to the subject an effective amount of a composition including EPA, DHA and lavender oil. In some instances, these methods include administering to the subject a composition in which the EPA and DHA are present in a 5:1 ratio.

In some of the methods of the present disclosure, the methods of supplementation described herein further include the step of identifying a subject in need of improvement of mental wellness and/or mood. In some of these methods, identifying a subject in need of improvement of focus and/or mental performance may include identifying a subject in need of at least one of improvement of mood, reduction of depression, increased energy or vigor, and/or improved self-esteem.

In some examples of the present disclosure the compositions may include approximately 1500 mg of fish oil per serving, with 975 mg of total omega-3 fatty acids. This may provide approximately 750 mg of EPA and approximately 150 mg of DHA with 75 mg of other omega-3s.

The present disclosure further provides nutritional supplements for improving mental wellness and/or mood in a subject. Such nutritional supplements may include an effective amount of a composition including EPA, DHA and lavender oil. In some instances, the supplements include a composition in which the EPA and DHA are present in a 5:1 ratio.

It should be noted that the methods described herein describe possible implementations, and that the operations and the steps may be rearranged or otherwise modified and that other implementations are possible. Further, aspects from two or more of the methods may be combined.

As used herein, including in the claims, “or” as used in a list of items (e.g., a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label, or other subsequent reference label.

The description set forth herein describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method of improving mental wellness and/or mood in a subject comprising:
administering to the subject an effective amount of a composition comprising;
eicosapentaenoic acid (“EPA”);
docosahexaenoic acid (“DHA”); and
lavender oil.

2. The method of claim 1, wherein the EPA and DHA are provided in a 5:1 ratio.

3. A method of improving mental wellness and/or mood in a subject comprising:
administering to the subject an effective amount of a composition comprising EPA and DHA in a 5:1 ratio; and
lavender oil.

4. A nutritional supplement comprising:
a 5:1 ratio of EPA and DHA; and
lavender oil.
